(19) United States
(12) Patent Application Publication
(10) Pub. No.: US 2020/0390517 A1
(43) Pub. Date: Dec. 17, 2020

(54) DYNAMIC HEAD, NECK AND BACK ASSIST APPARATUS WITH DAMPENING MECHANISM

(71) Applicants: Elias Zeilah, McLean, VA (US); Peter Alexander Murphy, Centreville, MD (US)

(72) Inventors: Elias Zeilah, McLean, VA (US); Peter Alexander Murphy, Centreville, MD (US)

(21) Appl. No.: 16/438,456

(22) Filed: Jun. 11, 2019

(51) Int. Cl. A61B 90/60; A61F 5/02; A61F 5/055; A61F 5/058; A61B 90/53

(52) U.S. Cl. CPC A61B 90/60; A61F 5/02; A61B 2090/502; A61F 5/05891; A61B 90/53; A61F 5/055

(57) ABSTRACT

A body brace is described. In some examples, a first member can be attached to a torso of a human body (to middle/lower portion of the body). The body brace can also include a second member attached to a head of the human body and arranged to support a chin of the human body. The body brace can further include a third member attached between the first member and the second member to distribute a force exerted between the first member and the second member.

### FIELD

Some implementations relate generally to a wearable mechanism for supporting a user's head, neck, shoulders, and/or back, and more particularly, to a dynamic head, neck and back assist apparatus with dampening mechanism.

### BACKGROUND

Surgeons, medical examiners and individuals with occupations requiring repetitive or prolonged neck extension on a regular basis engage their necks in a fixed upward or downward position for prolonged periods of time. Maintaining neck position in such positions for prolonged periods of time can cause pain and stiffness in the neck and shoulder muscles and general fatigue. Magnifying loupes used in the aforementioned occupations may require users to maintain their neck position within a very small window of movement in order to achieve the magnification factor of the lens. This limitation further increases the strain exerted on neck and shoulder muscles. In the cases where procedures last for several hours, this strain causes significant discomfort.

While engaging in activities with the neck in a fixed upward or downward position, the following muscles are subject to strain and/or injury: scalene, levator scapulae and suboccipital muscles. These muscles are engaged when rotating a user's neck and holding a neck position. Also engaged are subscapularis muscles in the shoulders and pectoralis minor muscles in the chest. Pain in any of these aforementioned areas could lead to fatigue and loss of dexterity, thereby significantly impacting a user's performance while executing such tasks in a leaning position.

There may be no existing solutions for supporting one's head, neck, or upper back during medical procedures and other tasks requiring the user's body to be in a leaning position for prolonged periods of time. Previous efforts to create a device that can assist a user in a leaning position only focused on supporting the lower body. These efforts failed to address the areas of the body most commonly affected when assuming a leaning position for long durations of time.

Such existing solutions for supporting a user's body while working in a leaning position consist of chairs and static neck braces. The former can be adjusted to suit the needs of the user. However, existing neck braces and chin supports lack the ability to be adjusted and secured in a manner that minimizes strain on one's head, neck, and upper back. There is no existing solution that assists the user in maintaining a head and neck position within a defined and fixed position.

It may be desirable to provide a new and/or improved head, neck and back assist system that can enable a user to maintain a head and neck position within a defined range of allowed positions for prolonged or extended periods of time. Some implementations were conceived in light of the above-mentioned limitations, needs, or problems, among other things.

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

### SUMMARY

Some implementations include a body support brace. The body support brace can include a first member attached to a torso of a human body. The body support brace can also include a second member attached to a head of the human body and arranged to support a chin of the human body. The body support brace can further include a third member attached between the first member and the second member to distribute a force exerted between the first member and the second member. The third member of the body support brace in this implementation can include a plurality of adjustable dampeners and can be configured to dampen forces exerted on one or more of the head, a neck, shoulders, back or torso of the human body.

In some implementations, the first member can include a washable, deformable, and/or adjustable material. The first member can also include one or more elastic fabric straps fitted around one or more of an upper or middle portion of the human body and can be fastened with one or more of one or more buckles, one or more friction belts, or adhered hook and loop fasteners. The first member can be fastened to the torso of the human body through one or more of a strap or hook and loop fasteners. In some implementations, the second member can include a washable, deformable, and adjustable chin support along with a washable, deformable, and adjustable head restraint. The second member can be fastened to the chin and the head via a strap attached around the head and secured using one or more hook and loop fasteners.

In some implementations, the third member can include a plurality of mechanisms to fasten the second member to the first member to transfer forces from the second member to the first member through the third member. The plurality of mechanisms can include a first locking mechanism disposed between the second member and the third member and a second locking mechanism disposed between the first member and the third member. The first locking mechanism can include a first rigid plate attached to the adjustable chin support and a first slot to fasten a first dampener of the third member to the first rigid plate. The second locking mechanism can include a second rigid plate attached to the first member and a second slot to fasten a second dampener of the third member to the second rigid plate.

In some implementations, the plurality of adjustable dampeners of the third member can be manually adjustable in length to accommodate a variable distance between the first member and the second member depending on body dimensions of a user wearing the body brace. The plurality of adjustable dampeners of the third member can also be manually adjustable in damping coefficients to support weight from the second member through the third member and in turn supported by the first member. The damping coefficients of the plurality of adjustable dampeners of the third member can be manually adjustable via an adjustable valve. Alternatively, damping coefficients of the plurality of adjustable dampeners of the third member can be automatically adjustable via one or more of electronically controlled switching valves or magnetorheological dampeners.

In some implementations, the plurality of adjustable dampeners of the third member can be locked in a fixed position to provide a defined range of motion of the second member with respect to the first member. In some implementations, the plurality of adjustable dampeners of the third member can be adjustable in configuration to provide a fixed range of motion of the second member with respect to the first member. In some implementations, the plurality of adjustable dampeners of the third member can be mounted inside a spring to enable compression and rebound of dampening assemblies within one or more of the plurality of adjustable dampeners.

In some implementations, the adjustable head restraint of the second member can include one or more of an adjustable strap secured around the head with a buckle, an adjustable friction belt, or an adjustable strap fastened around the head with one or more adhered hook and loop fasteners. In some implementations, the adjustable chin support of the second member can include a gel pad adhered to a rigid bottom member of the adjustable chin support. In some implementations, the adjustable head restraint of the second member can include a gel pad adhered to a rigid backing of the adjustable head restraint.

In some implementations, a method of supporting one or more of a neck, a head, shoulders, or a back of a human body can include attaching a first member to one or more of lower or middle portion of the human body. The method can also include attaching a second member to the head of the human body and to support a chin of the human body. The method can further include attaching a third member in between the first member and the second member. In this implementation, the third member can be configured to distribute a force exerted between the first member and the second member.

In some implementations, the first member can include a washable, deformable, and adjustable material. In some implementations, the second member can include a washable, deformable, and adjustable chin support along with a washable, deformable, and adjustable head restraint. In some implementations, the third member can include a plurality of adjustable dampeners and can be configured to dampen forces exerted on one or more of the head, the neck, the shoulders, the back or torso of the human body.

### BRIEF DESCRIPTION OF THE DRAWINGS

FIG. 1A is an elevated front perspective view of an exemplary body support brace in accordance with some implementations.

FIG. 1B is an elevated front perspective view of an exemplary body support brace with additional side dampers in accordance with some implementations.

FIG. 2A is an elevated side perspective view of an exemplary body support brace in accordance with some implementations.

FIG. 2B is an elevated side perspective view of an exemplary body support brace with additional side dampers in accordance with some implementations.

FIG. 3A is an elevated rear perspective view of an exemplary body support brace in accordance with some implementations.

FIG. 3B is an elevated rear perspective view of an exemplary body support brace with additional side dampers in accordance with some implementations.

FIG. 4A is an elevated front isometric perspective view of an exemplary body support brace in accordance with some implementations.

FIG. 4B is an elevated front isometric view of an exemplary body support brace with additional side dampers in accordance with some implementations.

FIG. 5A is an elevated top perspective view of an exemplary body support brace in accordance with some implementations.

FIG. 5B is an elevated top perspective view of an exemplary body support brace with additional side dampers in accordance with some implementations.

FIG. 6A is an elevated rear isometric perspective view of an exemplary body support brace in accordance with some implementations.

### DETAILED DESCRIPTION

Some implementations can include a wearable device supporting a user's head, neck, and/or back, based on a user's requirement, by distributing gravitational forces exerted on the head or the upper body through a system of supporting or dampening apparatuses to relieve strain on muscles in the aforementioned areas of the body when performing tasks requiring the user's body to be in a leaning position (forward/backward) for prolonged or extended periods of time.

The configurations of the disclosed subject matter described herein are not intended to represent the only configurations in which the disclosed subject matter may be practiced. This description includes specific details for the purpose of providing a thorough understanding of the disclosed subject matter. However, it may be apparent to those skilled in the art that the disclosed subject matter may be practiced without these specific details. In some instances, well known structures and components are shown in abstract form to avoid obscuring key concepts of the disclosure. Various embodiments described in the present disclosure may be carried out in different ways, forms and/or variations and/or in accordance with a desired application or implementation.

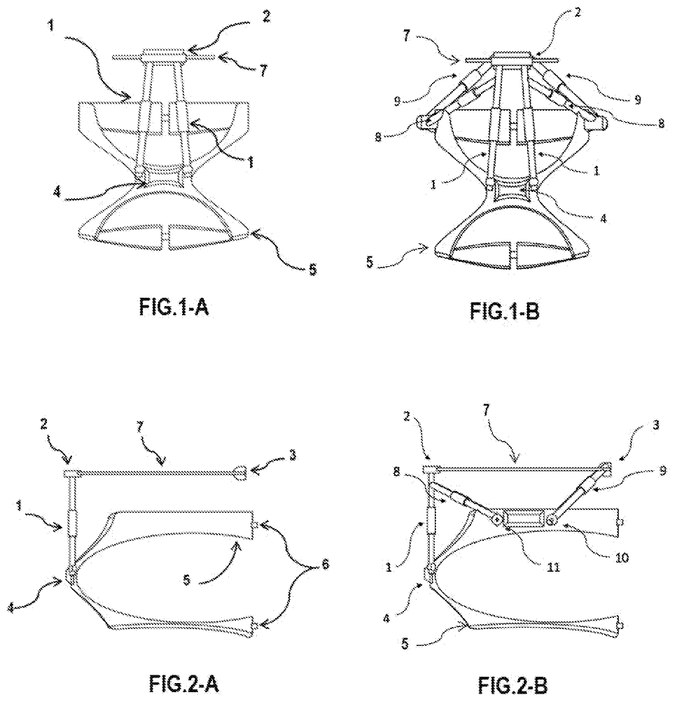

FIGS. 1 to 6 show diagrams of an example dynamic head, neck and back assist apparatus with dampening mechanism. Such a dynamic head, neck and back assist with dampening can, for example, be used by professionals such as surgeons, medical examiners and those in professions requiring repetitive and/or prolonged neck extension in a fixed upward or downward position.

FIG. 1A shows an exemplary dynamic head, neck and back assist apparatus with dampening mechanism 100. The dampening mechanism 100 can be of various sizes and/or shapes. The dampening mechanism can be made of a material that allows for adjustable dampening of forces due to gravitational effects as well as an adjustable rebound due to normal forces. However, any suitable material may be used. The dampening mechanism 100 can be capable of compressing and adjusting downward when a downward pressing force from the chin is applied, and then maintaining a fixed and supporting position. The dampening mechanism 100 can be capable of adjusting upward when upward tensioning force from the chin is applied and then maintaining a fixed and supporting position.

A support 102 can be made of an adjustable, deformable, and/or washable material composed of, for example, a soft gel material that allows for comfortable support to the human body. 102 can be made in such a way so as to allow for a fixed attachment around the torso of the human body. Another support 104 can be mounted on the front side of 102 in a fashion that allows for comfortable attachment of the dampening mechanism 100.

A rigid center support piece 106 can be made of a rigid material. The rigid center support piece 106 can include one or more dampeners and can be configured to dampen forces exerted on one or more of the head, the neck, the shoulders, the back or torso of the human body. The support 104 can be made to allow for the one or more dampeners to be connected to and supported by the rigid center support piece 106. The support 104 can be embedded within yet another support 108 that can be made of a washable, deformable, and adjustable material.

Also, the support 108 can allow for the bottom end of the dampening mechanism 100 to be supported by the body. Further, the support 108 can be attached to the torso of the human body, 102 can be attached to the head of the human body and arranged to support the chin of the human body, and the rigid center support piece 106 can be attached between the support 102 and the support 108 to distribute a force exerted from the support 102 to the support 108 or between the support 102 and the support 108.

FIG. 2A shows a chin support or a chin brace 202, which can be of different sizes and/or shapes. The chin support or chin brace 202 can be made of a material composed of a rigid bottom that is covered with washable liner. The washable liner can be made of a soft gel material to allow for comfortable bracing of the chin over extended periods of time. FIG. 2A also shows a head restraint or head brace 204, which can also be of different sizes and/or shapes. The head restraint or head brace 204 can be made of a material composed of a rigid backing covered with a washable liner made of a soft gel material to allow for comfortable bracing of the back of the head over extended periods of time. FIG. 2A also shows a connecting band 206, which can be made of a material that is flexible/adjustable to fit different head sizes. Both sides of the chin support 202 can be attached to the connecting band 206, which is attached to the head restraint 204 on both sides. The chin support 202 and the head restraint 204, connected via the connecting band 206, can fit firmly around the head to support both the chin and the back of the head. The chin support 202 is connected to a dampening mechanism 100 in such a way that the size of the dampening mechanism 100 can be adjustable. The chin support 202 and/or the head restraint 204 can be washable, deformable, and/or adjustable.

FIG. 2B shows a forward side leaning dampening device 208, which can be of different sizes and/or shapes. The forward side leaning dampening device 208 can be made of a material that allows for adjustable dampening of forces due to gravitational effects as well as adjustable rebound due to normal forces. However, any other suitable material may be used. FIG. 2B also shows a rear side leaning dampening device 210, which can be of different sizes and/or shapes. The rear side leaning dampening device 210 can be made of a material that allows for adjustable dampening of forces due to gravitational effects as well as adjustable rebound due to normal forces. However, any other suitable material may be used. The forward side leaning dampening device 208 and the rear side leaning dampening device 210 can be adjustable in length to accommodate a variable distance between 102 and 106, 106 and 108, and/or 102 and 108 depending on the body dimensions of a user wearing the body brace.

Support 106 can include a plurality of mechanisms to fasten support 102 to support 108 to transfer forces from 108 to 102 through 106. FIG. 2B also shows a support 212 that can be mounted on the left side and the right side of support 102 in a manner that allows for comfortable attachment of 208 to the dampening mechanism 100. FIG. 2B also shows a support 214 that can be mounted on the left side and the right side of support 102 in a fashion that allows for comfortable attachment of 210 to the dampening mechanism 100. 208 and 210 can be adjustable in damping coefficients to support weight from 102, 106 and/or 108. Damping coefficients of 208 and 210 can be manually adjustable via an adjustable valve or automatically adjustable via one or more of electronically controlled switching valves or magnetorheological dampeners.

In an example implementation, 208 and 210 can be locked in a fixed position to provide a defined range of motion of 102 with respect to 108. In another example implementation, 208 and 210 can be adjustable in configuration to provide a fixed range of motion of 102 with respect to 108. In some implementations, 208 and 210 can be mounted inside a spring to enable compression and rebound of dampening assemblies within 208 and 210 respectively. In some other implementations, the head restraint 204 can include one or more of an adjustable strap secured around the head with a buckle, an adjustable friction belt, or an adjustable strap fastened around the head with one or more adhered hook and loop fasteners. In one implementation, support 102 can be fastened to the chin and the head via a strap attached around the head and secured using one or more hook and loop fasteners while support 108 can be fastened to the torso of the human body through one or more of a strap or hook and loop fasteners.

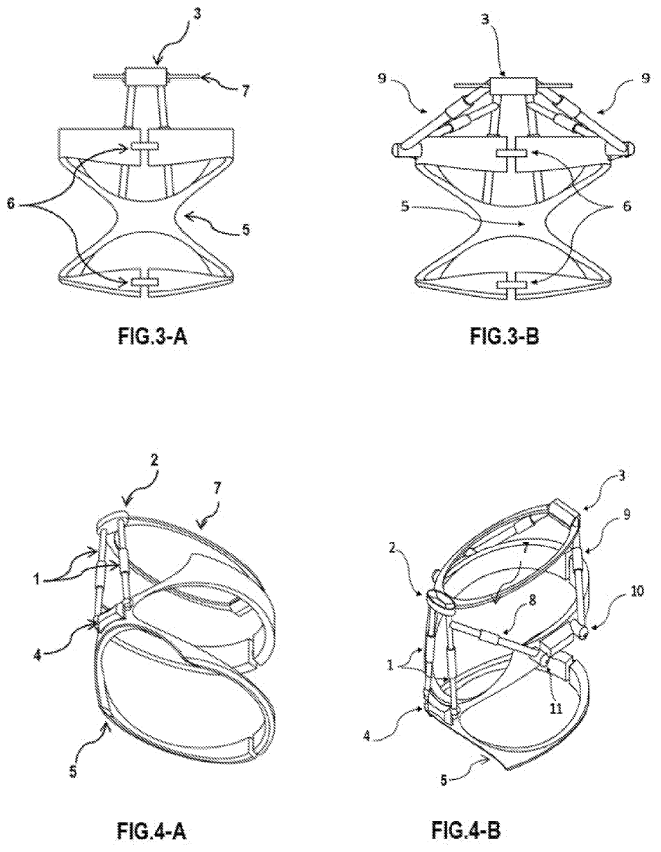

FIG. 3A shows a securing mechanism 302 that can be made from a material allowing for comfortable securement of both back sections of support 102. Mechanism 302 can also be made in a manner that allows for support 102 to be adjustable so as to be secured to various body sizes. FIG. 3B shows the body support brace with additional side dampers in some implementations. FIG. 5A shows an elevated top perspective view of the body support brace in accordance with some implementations. FIG. 5B shows an elevated top perspective view of the body support brace with additional side dampers in accordance with some implementations.

In some implementations, the support 108 can include one or more elastic fabric straps fitted around one or more of an upper or middle portion of the human body and can be fastened with one or more of one or more buckles, one or more friction belts, or adhered hook and loop fasteners. In some other implementations, support 106 can include a plurality of mechanisms to fasten support 102 to support 108 to transfer one or more forces from 102 to 108 through 106. The plurality of mechanisms can include a first locking mechanism disposed between 102 and 106 and a second locking mechanism disposed between 108 and 106. The first locking mechanism can include a first rigid plate attached to the chin support 202 and a slot to fasten a dampener of 106 to the first rigid plate. The second locking mechanism can include a second rigid plate attached to 108 and a slot to fasten another dampener of 106 to the second rigid plate.

An example process/method for assisting a user's head, neck, shoulders and/or back of a human body can include attaching support 108 to one or more of a lower or middle portion of the human body. Another step can include attaching support 102 to the head of the human body and to support a chin of the human body. The method can further include attaching support 106 in between support 108 and support 102. Support 106 can be configured to distribute a force exerted between support 102 and support 108.

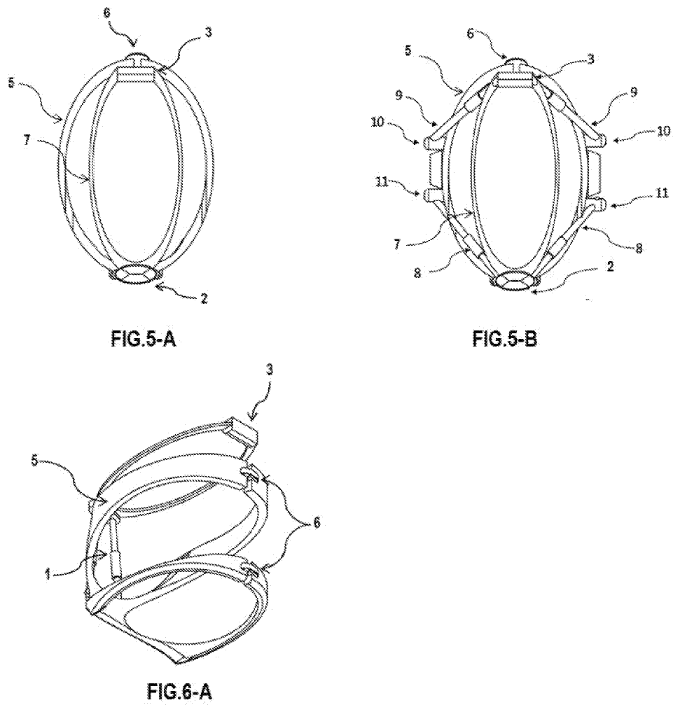

FIG. 4A shows an elevated front isometric perspective view of the body support brace in some implementations. FIG. 4B shows an elevated front isometric view of the body support brace with additional side dampers in some implementations. FIG. 6A shows an elevated rear isometric perspective view of the body support brace in some implementations.

The present disclosure is described to enable any person skilled in the art to practice the various aspects described herein. The disclosure provides various examples of the disclosed invention, but the disclosure is not limited to these examples. Various modifications to these aspects will be readily apparent to those skilled in the art, and the generic principles defined herein may be applied to other aspects.

The dynamic head, neck and back assist with dampening can be a device in some implementations. It will be appreciated that the dynamic head, neck and back assist with dampening described herein is for illustration purposes only and is not intended to be limiting. Other dynamic head, neck and back assist with dampening mechanisms may be used depending on a contemplated implementation. It will be appreciated that supports, braces, and dampeners described herein are for illustration purposes only and are not intended to be limiting. Other types of supports/braces/dampeners may be used depending on a contemplated implementation.

It will be appreciated that any dimensions described herein are for illustration purposes only and are not intended to be limiting. Other dimensions could be used depending on a contemplated implementation.

It is therefore apparent that there are provided, in accordance with the various example implementations disclosed herein, a dynamic head, neck and back assist with dampening as well as a method for supporting a user's head, neck, shoulders or back when a user's body is required to be in a leaning position (forward or backward) for prolonged and/or extended periods of time.

While the disclosed subject matter has been described in conjunction with a number of implementations, it is evident that many alternatives, modifications and variations would be or are apparent to those of ordinary skill in the applicable arts. Accordingly, Applicant intends to embrace all such alternatives, modifications, equivalents and variations that are within the spirit and scope of the disclosed subject matter.

What is claimed is:

1. A body support brace comprising:
a first member attached to a torso of a human body;
a second member attached to a head of the human body and arranged to support a chin of the human body; and
a third member attached between the first member and the second member to distribute a force exerted between the first member and the second member.

2. The body support brace of claim 1, wherein the first member comprises a washable, deformable, and adjustable material, and wherein the second member comprises a washable, deformable, and adjustable chin support along with a washable, deformable, and adjustable head restraint, and wherein the third member comprises a plurality of adjustable dampeners and is configured to dampen forces exerted on one or more of the head, a neck, shoulders, back or torso of the human body.

3. The body support brace of claim 2, wherein the third member further comprises a plurality of mechanisms to fasten the second member to the first member to transfer forces from the second member to the first member through the third member.

4. The body support brace of claim 1, wherein the second member is fastened to the chin and the head via a strap attached around the head and secured using one or more hook and loop fasteners, and wherein the first member is fastened to the torso of the human body through one or more of a strap or hook and loop fasteners.

5. The body support brace of claim 3, wherein the plurality of mechanisms includes a first locking mechanism disposed between the second member and the third member, the first locking mechanism comprising a first rigid plate attached to the adjustable chin support and a first slot to fasten a first dampener of the third member to the first rigid plate, and a second locking mechanism disposed between the first member and the third member, the second locking mechanism comprising a second rigid plate attached to the first member and a second slot to fasten a second dampener of the third member to the second rigid plate.

6. The body support brace of claim 2, wherein the plurality of adjustable dampeners of the third member are adjustable in length to accommodate a variable distance between the first member and the second member depending on body dimensions of a user wearing the body support brace.

7. The body support brace of claim 2, wherein the plurality of adjustable dampeners of the third member include adjustable damping coefficients to support weight from the second member through the third member and in turn supported by the first member.

8. The body support brace of claim 7, wherein the adjustable damping coefficients of the plurality of adjustable dampeners of the third member are manually adjustable via an adjustable valve.

9. The body support brace of claim 7, wherein the adjustable damping coefficients of the plurality of adjustable dampeners of the third member are automatically adjustable via one or more of electronically controlled switching valves or magnetorheological dampeners.

10. The body support brace of claim 2, wherein the plurality of adjustable dampeners of the third member are locked in a fixed position to provide a defined range of motion of the second member with respect to the first member.

11. The body support brace of claim 2, wherein the plurality of adjustable dampeners of the third member are adjustable in configuration to provide a fixed range of motion of the second member with respect to the first member.

12. The body support brace of claim 2, wherein the adjustable head restraint of the second member comprises one or more of an adjustable strap secured around the head with a buckle, an adjustable friction belt, or an adjustable strap fastened around the head with one or more adhered hook and loop fasteners.

13. The body support brace of claim 2, wherein the first member comprises one or more elastic fabric straps fitted around one or more of an upper or middle portion of the human body and is fastened with one or more of one or more buckles, one or more friction belts, or adhered hook and loop fasteners.

14. The body support brace of claim 2, wherein the adjustable chin support of the second member comprises a gel pad adhered to a rigid bottom member of the adjustable chin support.

15. The body support brace of claim 2, wherein the adjustable head restraint of the second member comprises a gel pad adhered to a rigid backing of the adjustable head restraint.

16. The body support brace of claim 2, wherein the plurality of adjustable dampeners of the third member are mounted inside a spring to enable compression and rebound of dampening assemblies within one or more of the plurality of adjustable dampeners.

17. A method of supporting one or more of a neck, a head, shoulders, or a back of a human body, the method comprising:
attaching a first member to one or more of lower or middle portion of the human body;
attaching a second member to the head of the human body and to support a chin of the human body; and
attaching a third member in between the first member and the second member, wherein the third member is configured to distribute a force exerted between the first member and the second member.

18. The method of claim 17, wherein the first member comprises a washable, deformable, and adjustable material.

19. The method of claim 17, wherein the second member comprises a washable, deformable, and adjustable chin support along with a washable, deformable, and adjustable head restraint.

20. The method of claim 17, wherein the third member comprises a plurality of adjustable dampeners and is configured to dampen forces exerted on one or more of the head, the neck, the shoulders, the back or torso of the human body.
